(19) United States
(12) Patent Application Publication
(10) Pub. No.: US 2019/0345651 A1
(43) Pub. Date: Nov. 14, 2019

(54) ANTI-SLIP SOCK

(71) Applicant: SUNPOLAR International Co., Ltd, Changhua County (TW)

(72) Inventor: SHOU HSIU LIANG, Changhua County (TW)

(21) Appl. No.: 15/978,251

(22) Filed: May 14, 2018

(51) Int. Cl. D04B 1/10; A41B 11/00; A61F 13/08; D04B 1/26; D04B 1/14

(52) U.S. Cl. CPC D04B 1/102; A41B 11/008; D04B 1/14; D04B 1/265; A61F 13/08

(57) ABSTRACT

An anti-slip sock includes a sock body configured to be worn on a human foot. The sock body woven from fabric yarn threads defines opposite outer and inner surfaces and has a sock sole. The sock sole corresponds to a sole of the foot with the inner surface facing the foot. The sock sole is woven from the fabric yarn threads together with anti-slip yarn threads. The sock sole is provided with a protective means that prevents the human foot from contacting the anti-slip yarn threads. Thereby, contact between the anti-slip yarn threads and human skin and in turn the risk of skin irritation can be eliminated.

### BACKGROUND OF THE INVENTION

### 1. Field of the Invention

The present invention relates to socks, and more particularly to an anti-slip sock.

### 2. Description of the Related Art

Medical stockings are known to prevent embolism or blood clots by applying human legs with progressive pressure that improves the rate of blood flow through veins. Most existing medical stockings having silicone coating at outer surface preventing slippage.

In the description about prior-art techniques in U.S. Pat. No. 6,673,421B1, some known processes for coating medical stockings with silicone are mentioned. In these known processes, silicone is first hot melted into liquid silicone and applied to the outer surface of stockings.

However, since yarn-woven stockings always have meshes, liquid silicone applied thereto tends to permeate through these meshes and be exposed at the inner surface of stockings.

Thus, when users wear such medical stockings, direct contact between users' skin and silicone can give tacky feeling and discomfort and long-term wear may even bring about skin irritation. Particularly, users with leg injury are more likely to suffer irritation and discomfort caused by silicone.

### SUMMARY OF THE INVENTION

To address the foregoing issue, the present invention provides an anti-slip sock with a sock sole woven from anti-slip yarn threads and fabric yarn threads. The anti-slip sock features a protective means that allows the anti-slip yarn threads to be exposed at the outer surface of the sock and eliminates the risk of direct contact between a human foot and the anti-slip yarn threads, thereby protecting its users from discomfort and irritation otherwise caused by the anti-slip yarn threads.

According to one embodiment of the present invention, the anti-slip sock comprises a sock body that is configured to be worn on a human foot and is woven from fabric yarn threads. The sock body defines opposite outer and inner surfaces. The sock body has a sock sole. When the sock is worn by a user, the sock sole corresponds to a sole of the user's foot and the inner surface faces the foot. The sock sole is woven from the fabric yarn threads and anti-slip yarn threads. Each of the anti-slip yarn threads has spaced anti-slip segments. The anti-slip segments are exposed at the outer surface. The sock sole has a protective means that prevents the user's foot from contacting the anti-slip yarn threads.

With the foregoing configuration, the sock sole woven from the fabric yarn threads and the anti-slip yarn threads is equipped with the protective means, so that when the sock body of the sock is worn on a human foot, contact between the foot and any of the anti-slip yarn threads is prevented. Thereby, the anti-slip yarn threads are exposed at the outer surface to provide anti-slip effect, without foot discomfort caused by direct contact between the foot and silicone coating as seen in the prior art socks and stockings.

Furthermore, the anti-slip yarn threads are woven into the sock sole and well secured from departing from the sock body, thereby ensuring endurable and reliable anti-slip effect.

### BRIEF DESCRIPTION OF THE DRAWINGS

FIG. 1 is a perspective view of an anti-slip sock of the present invention.

FIG. 2 is a back view of the anti-slip sock of FIG. 1.

FIG. 3 is a cross-sectional view of the sock sole taken along Line 3-3 of FIG. 2, showing that anti-slip segments are level with outside segments.

FIG. 4 is a cross-sectional view of the sock sole according to a second embodiment of the present invention, showing that the anti-slip segments raised from the outer surface.

FIG. 5 is a cross-sectional view of the sock sole according to a third embodiment of the present invention, showing that the inner surface is provided with a protective sheet, and the threading segments are level with the inside segments.

FIG. 6 is a cross-sectional view of the sock sole according to a fourth embodiment of the present invention, showing that the inner surface is provided with a protective sheet, and the threading segments jut out from the inside segments.

FIG. 7 is a cross-sectional view of the sock sole according to a fifth embodiment of the present invention, showing that the inner surface is provided with a protective sheet, and the threading segments are raised from the inner surface.

FIG. 8 is an applied view of the anti-slip sock, wherein its inner surface is provided with a protective sheet, and the anti-slip segments are raised from the outer surface.

### DETAILED DESCRIPTION OF THE INVENTION

The following preferred embodiments when read with the accompanying drawings are made to clearly exhibit the above-mentioned and other technical contents, features and effects of the present invention. Through the exposition by means of the specific embodiments, people would further understand the technical means and effects the present invention adopts to achieve the above-indicated objectives. However, the accompanying drawings are intended for reference and illustration, but not to limit the present invention and are not made to scale.

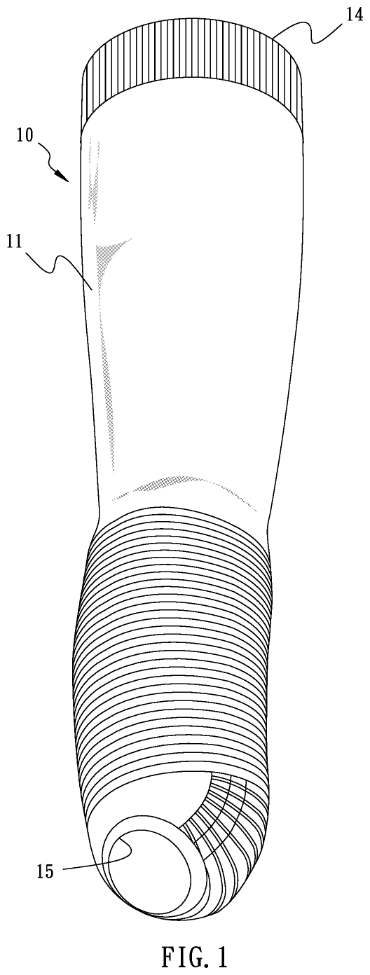

FIG. 1 through FIG. 3 and FIG. 8 illustrate an anti-slip sock according to a first embodiment of the present invention, whose configuration will be described below.

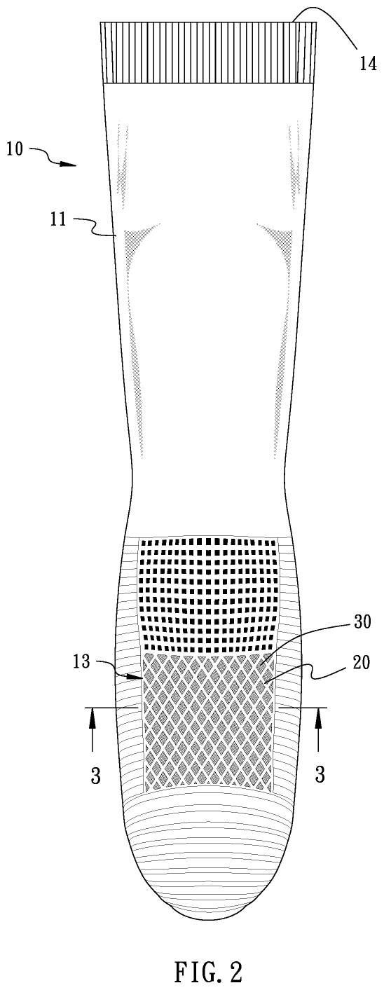

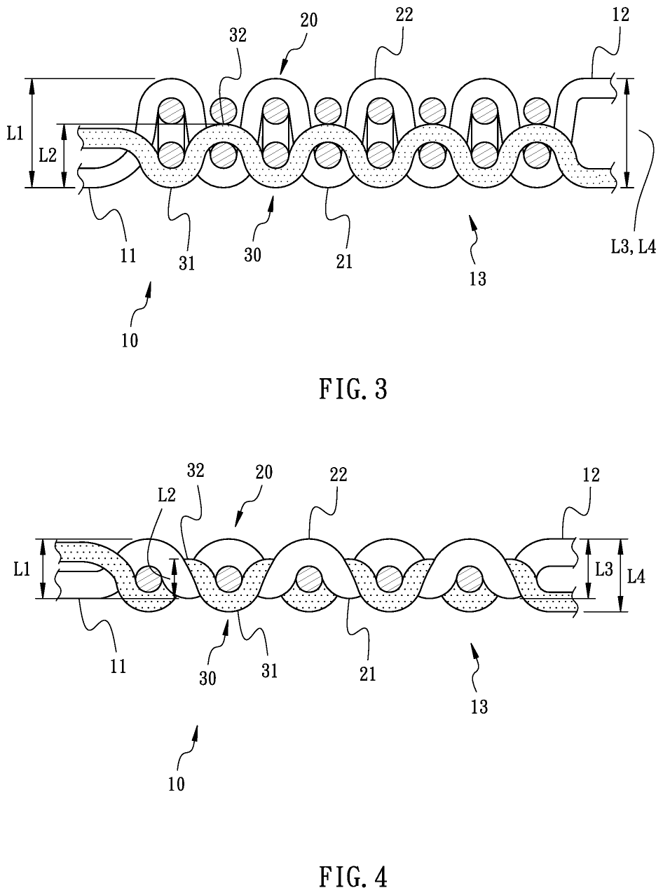

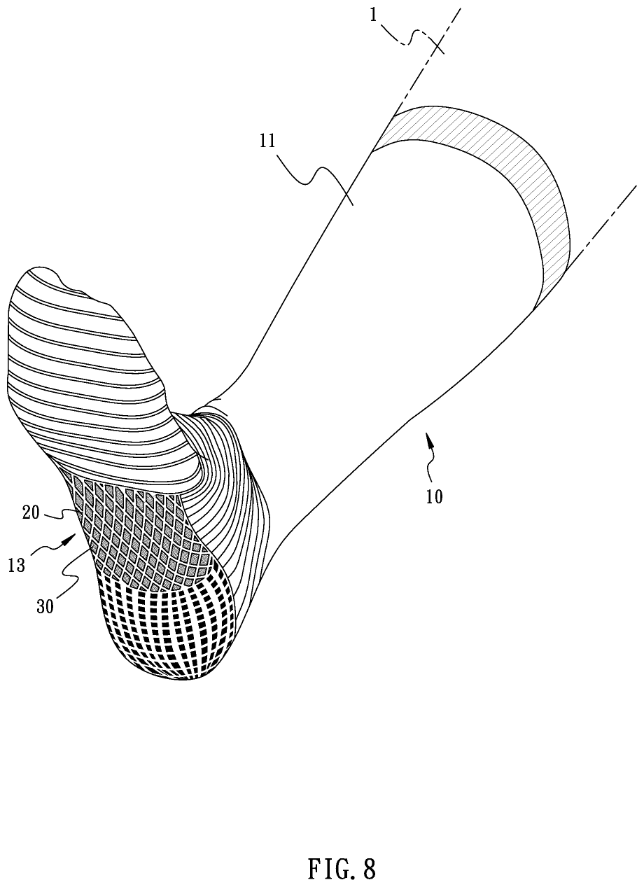

The anti-slip sock has a tubular sock body 10 to be worn on a human foot 1. The sock body 10 woven from fabric yarn threads 20 defines an outer surface 11 and an inner surface 12 opposite to each other, and has a sock sole 13. The sock body 10 has two ends thereof formed with a first hole 14 and a second hole 15, respectively. Referring to FIG. 2, FIG. 3 and FIG. 8, when a human foot 1 enters the sock body 10 through the first hole 14, the second hole 15 of the sock body 10 corresponds to toes of the foot 1, while the toes do not stick out from the second hole 15, and the sock sole 13 corresponds to a sole of the foot 1. At this time, the inner surface 12 of the sock body 10 faces the foot 1, and the outer surface 11 of the sock body 10 faces the outside.

The sock sole 13 is woven from fabric yarn threads 20 and anti-slip yarn threads 30. The sock sole 13 uses a protective means to prevent the human foot 1 wearing the sock body 10 from contacting any of the anti-slip yarn threads 30.

The protective means is realized as below. The fabric yarn threads 20 each have alternately arranged outside segments 21 and inside segments 22, and the anti-slip yarn threads 30 each have alternately arranged anti-slip segment 31 and threading segments 32. Each of the threading segments 32 is sandwiched between the outside segment 21 and the inside segment 22. The anti-slip segment 31 has a length greater than or equal to that of the threading segment 32. The anti-slip yarn threads 30 are exposed at the outer surface 11. Therein, the area of each of the anti-slip yarn threads 30 exposed at the outer surface 11 of the sock sole 13 is greater than the area of each of the fabric yarn threads 20 exposed at the outer surface 11 of the sock sole 13. In the embodiment, the anti-slip yarn threads 30 are silicone threads.

Additionally, the vertical distance between the inside segments 22 and the outer surface 11 is defined as a first interval L1, and the vertical distance between the threading segments 32 and the outer surface 11 is defined as a second interval L2. The first interval L1 is greater than the second interval L2. The vertical distance between the outside segments 21 and the inner surface 12 is defined as a third interval L3, and the vertical distance between the anti-slip segment 31 and the inner surface 12 is defined as a fourth interval L4. Referring to FIG. 3, in the present embodiment, the length of each of the anti-slip segment 31 is equal to the length of each of the threading segments 32. The third interval L3 is equal to the fourth interval L4, and the outside segments 21 and the anti-slip segment 31 are level with the outer surface 11. As a result, the outer surface 11 corresponding to the sock sole 13 is flat.

The sock sole 13 of the sock body 10 is woven from the fabric yarn threads 20 and the anti-slip yarn threads 30, and implements the protective means to prevent a human foot 1 from contacting any of the anti-slip yarn threads 30. Thereby, anti-slip effect is ensured by the anti-slip yarn threads 30 exposed at the outer surface 11, while the risk of skin irritation caused by direct contact between the anti-slip yarn threads 30 and the foot 1 is eliminated.

In addition, the anti-slip yarn threads 30 are interlaced with the fabric yarn threads 20 to form the sock sole 13, and secured from departing from the sock body 10, so as to ensure the anti-slip effect of the sock body 10.

FIG. 4 depicts a second embodiment of the present invention, which is different from the first embodiment for having the following distinctive features.

In the second embodiment of the present invention, the length of each of the anti-slip segments 31 is equal to the length of each of the threading segments 32. The first interval L1 is greater than the second interval L2. The third interval L3 is smaller than the fourth interval L4. Each of the anti-slip segment 31 is farther from the outer surface 11 than each of the outside segments 21 is. In other words, each of the anti-slip segment 31 is raised from the outer surface 11, and the outer surface 11 corresponding to the sock sole 13 is uneven.

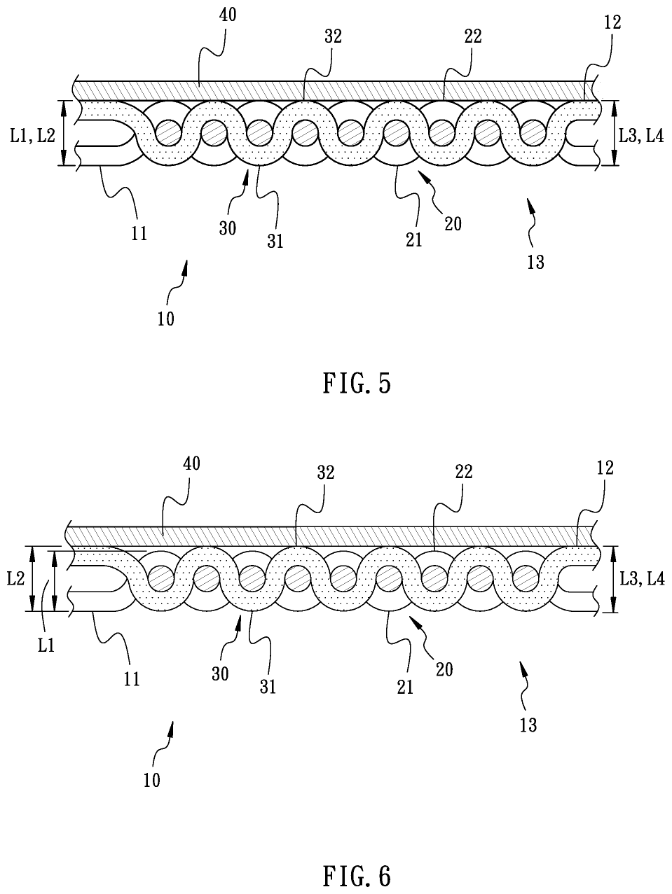

FIG. 5 depicts a third embodiment of the present invention, which is different from the first embodiment for having the following distinctive features

In the third embodiment of the present invention, the protective means further comprises a protective sheet 40. The protective sheet 40 is adhered to or woven into the inner surface 12. The protective sheet 40 covers the threading segments 32, so as to prevent the human foot 1 from contacting the anti-slip yarn threads 30. Therein, the length of each of the anti-slip segment 31 is greater than the length of each of the threading segments 32. The first interval L1 is equal to the second interval L2. The third interval L3 is equal to the fourth interval L4. Each of the outside segments 21 and each of the anti-slip segments 31 are level with the outer surface 11. Each of the inside segments 22 and each of the threading segments 32 are level with the inner surface 12, so that the outer surface 11 and the inner surface 12 corresponding to sock sole 13 are even.

FIG. 6 depicts a fourth embodiment of the present invention, which is different from the first embodiment for having the following distinctive features.

In the fourth embodiment of the present invention, the length of each of the anti-slip segment 31 is greater than the length of each of the threading segments 32. The first interval L1 is smaller than the second interval L2. The third interval L3 is equal to the fourth interval L4. Each of the outside segments 21 and each of the anti-slip segments 31 are level with the outer surface 11. Each of the inside segments 22 is farther away from the inner surface 12 than each of the threading segments 32 is. Thereby, the inner surface 12 corresponding to the sock sole 13 is uneven.

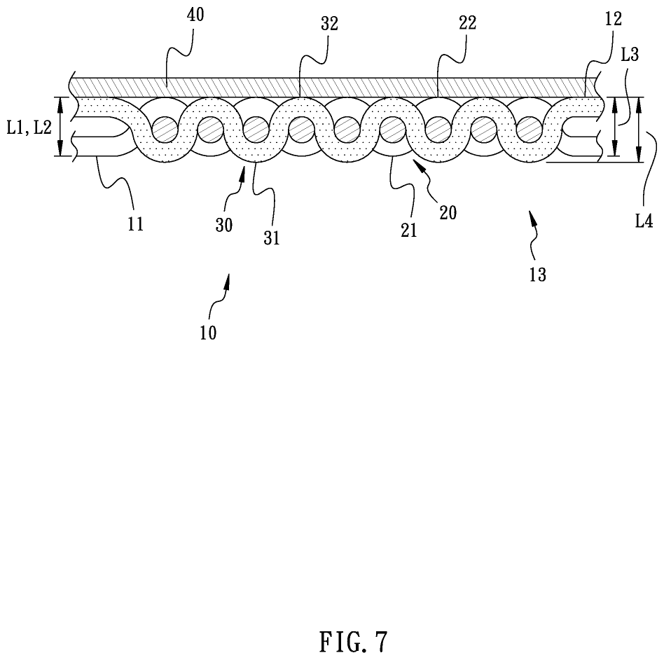

FIG. 7 depicts a fifth embodiment of the present invention, which is different from the first embodiment for having the following distinctive features.

In the fifth embodiment of the present invention, the length of each of the anti-slip segment 31 is greater than the length of each of the threading segments 32. The first interval L1 is equal to the second interval L2. The third interval L3 is smaller than the fourth interval L4. Each of the inside segments 22 and each of the threading segments 32 are level with the inner surface 12. Each of the anti-slip segment 31 is farther away from the outer surface 11 than each of the outside segments 21 is. In other words, each of the anti-slip segment 31 is raised from the outer surface 11, and thereby the inner surface 12 corresponding to the sock sole 13 is even while the outer surface 11 corresponding to the sock sole 13 is uneven.

To sum up, the present invention has the following beneficial effects.

First, the sock sole 13 of the sock body 10 is woven from the fabric yarn threads 20 and the anti-slip yarn threads 30 so that each of the anti-slip yarn threads 30 is exposed at the outer surface 11 of the sock body 10, and the protective means prevents a human foot 1 put in the sock body 10 from contacting any of the anti-slip yarn threads 30. Thereby, anti-slip effect is ensured by the anti-slip yarn threads 30 exposed at the outer surface 11, while the risk of skin irritation caused by direct contact between the anti-slip yarn threads 30 and the foot 1 is eliminated.

Second, the anti-slip yarn threads 30 are interlaced with the fabric yarn threads 20 to form the sock sole 13, and secured from departing from the sock body 10, so as to ensure the anti-slip effect of the sock body 10.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. An anti-slip sock, comprising:
a sock body, configured to be worn on a human foot, being woven from fabric yarn threads, defining opposite outer and inner surfaces, and having a sock sole, wherein when worn, the sock has the sock sole corresponding to a sole of the foot and has the inner surface facing the foot, wherein the sock sole is woven from the fabric yarn threads together with the anti-slip yarn threads, in which each the anti-slip yarn thread has spaced anti-slip segments that are exposed at the outer surface, and the sock sole has a protective means to prevent direct contact between the foot and the anti-slip yarn threads.

2. The anti-slip sock of claim 1, wherein the protective means comprises the fabric yarn threads that each has alternately arranged outside segments and inside segments, and the anti-slip yarn threads that each has the alternately arranged anti-slip segments and the threading segments, in which each the threading segment is sandwiched between the one outside segment and the one inside segment.

3. The anti-slip sock of claim 2, wherein a vertical distance between the inside segments and the outer surface is a first interval, and a vertical distance between the threading segments and the outer surface is a second interval, in which the first interval is greater than the second interval.

4. The anti-slip sock of claim 2, wherein a vertical distance between the outside segments and the inner surface is a third interval, and a vertical distance between the anti-slip segments and the inner surface is a fourth interval, in which the third interval is smaller than or equal to the fourth interval.

5. The anti-slip sock of claim 4, wherein each the anti-slip segment has a length greater than or equal to a length of each the threading segment.

6. The anti-slip sock of claim 1, wherein the protective means comprises a protective sheet that is adhered to or woven into the inner surface.

7. The anti-slip sock of claim 6, wherein the protective means further comprises the fabric yarn threads that each has the alternately arranged outside segments and the inside segments, and the anti-slip yarn threads that each has the alternately arranged anti-slip segments and the threading segments, in which each the threading segment is sandwiched between the one outside segment and the one inside segment, and the protective sheet covers the threading segments.

8. The anti-slip sock of claim 7, wherein a vertical distance between the inside segments and the outer surface is a first interval, and a vertical distance between the threading segments and the outer surface is a second interval, in which the first interval is equal to or smaller than the second interval.

9. The anti-slip sock of claim 8, wherein vertical distance between the outside segments and the inner surface is a third interval, and a vertical distance between the anti-slip segments and the inner surface is a fourth interval, in which the third interval is smaller than or equal to the fourth interval.

10. The anti-slip sock of claim 9, wherein each the anti-slip segment has a length greater than or equal to a length of each the threading segment.
